(19) United States
(12) Patent Application Publication
(10) Pub. No.: US 2004/0048376 A1
(43) Pub. Date: Mar. 11, 2004

(54) Methods for modulating splicing and/or alternative splicing, and for identifying alternatively spliced units in genes

(76) Inventors: Benoit Chabot, Sherbrooke (CA); Lucie Bolduc, East Angus (CA)

Correspondence Address: GOUDREAU GAGE DUBUC, 800 PLACE VICTORIA, SUITE 3400, MONTREAL, QUEBEC, H4Z 1E9, CA

(21) Appl. No.: 09/832,183

(22) Filed: Apr. 10, 2001

(30) Foreign Application Priority Data
Apr. 10, 2000 (CA) 2,305,956

(51) Int. Cl. C12N 15/85

(52) U.S. Cl. 435/455

(57) ABSTRACT

The present invention relates to splicing and especially to alternative RNA splicing which is involved in the production of protein isoforms with distinct activities. More specifically, the present invention relates to methods for modulating alternative splicing, and for identifying alternatively spliced units in genes. The present invention also concerns methods for modulating the ratio of alternatively spliced isoforms relative to each other and to normalize the alternative splicing actions of a splicing extract. The invention also relates to kits for normalizing and/or modulating splicing and/or alternative splicing of transcripts. More particularly the invention relates to a method to normalize a splicing and/or alternative splicing activity of an extract comprising an addition thereto of an effective amount of a polar aprotic solvent, whereby the effective amount normalizes splicing and/or alternative splicing as compared to an untreated extract. Examples of polar aprotic solvents of the invention include DMSO, DMF, formamide, HMPA, N-methylformamide, nitromethane, acetone, and acetonitrile.

### FIELD OF THE INVENTION

The present invention relates to splicing and especially to alternative RNA splicing which is involved in the production of protein isoforms with distinct activities. More specifically, the present invention relates to methods for modulating alternative splicing, and for identifying alternatively spliced units in genes. The present invention also concerns methods for modulating the ratio of alternatively spliced isoforms relative to each other and to normalize the alternative splicing actions of a splicing extract. The invention also relates to kits for normalizing and/or modulating splicing and/or alternative splicing of transcripts.

### BACKGROUND OF THE INVENTION

Alternative splicing is a process which involves the selective use of splice sites on a mRNA precursor. Alternative splicing allows the production of many proteins from a single gene and therefore allows the generation of proteins with distinct functions. Alternative splicing events can occur through a variety of ways including exon skipping, the use of mutually exclusive exons and the differential selection of 5′ and/or 3′ splice sites. For many genes (e.g., homeogenes, oncogenes, neuropeptides, extracellular matrix proteins, muscle contractile proteins), alternative splicing is regulated in a developmental or tissue-specific fashion. Alternative splicing therefore plays a critical role in gene expression. Recent studies have revealed the importance of alternative splicing in the expression strategies of complex organisms. It is estimated that at least 35%, and probably more than 50%, of all the human genes are alternatively spliced. Since many genes have more than two, and some have the potential to have up to several thousand alternatively spliced mRNA isoforms, the identity of more than 95% of the total number of human proteins may be determined by alternative splicing of mRNA precursors. While the implication of alternative splicing and its regulation on cellular function has been recognized, its precise contribution to fundamental cellular processes is still embryonic. There thus remains a need to identify and characterize new alternative splicing units.

Alternative splicing of mRNA precursors (pre-mRNAs) plays an important role in the regulation of mammalian gene expression. The regulation of alternative splicing occurs in cells of various lineages and is part of the expression program of a large number of genes. Recently, it has become clear that alternative splicing controls the production of proteins isoforms which, sometimes, have completely different functions. Oncogene and proto-oncogene protein isoforms with different and sometimes antagonistic properties on cell transformation are produced via alternative splicing. Examples of this kind are found in Makela, T. P. et al. 1992, Science 256:373; Yen, J. et al. 1991, Proc. Natl. Acad. Sci. U.S.A. 88:5077; Mumberg, D. et al. 1991, Genes Dev. 5:1212; Foulkes, N. S. and Sassone-Corsi, P. 1992, Cell 68:411. Also, alternative splicing is often used to control the production of proteins involved in programmed cell death such as Fas, Bcl-2, Bax, and Ced-4 (Jiang, Z. H. and Wu J. Y., 1999, Proc Soc Exp Biol Med 220: 64). Alternative splicing of a pre-mRNA may produce a repressor protein, while an activator may be produced from the same pre-mRNA in different conditions (Black D. L. 2000, Cell 103:367; Graveley, B. R. 2001, Trends Genet. 17:100).

While a lot of efforts are now devoted to the understanding of how splicing regulation is achieved in mammalian cells (Chabot, B. 1996, Trends Genet. 12:472) and despite the biological relevance of alternative splicing to cell growth, cell differentiation and mammalian development, a detailed understanding of the process is still lacking. In most cases, the nature of regulatory elements, the identity of trans-acting factors and the mechanisms involved in the regulation remain unknown. Thus, there remains a need to better understand the relevance of alternative splicing to cell growth, cell differentiation and mammalian development. There also remains a need to better understand how splicing and particularly alternative splicing and the regulation thereof are controlled in cells. In addition, there remains a need to identify agents which can modulate alternative splicing in cells and animals.

Several protein splicing regulators affect the initial ATP-independent steps of spliceosome assembly, which include the recognition of the 5′ splice site by U1 snRNP and the recognition of the polypyrimidine tract/3′ splice site by U2AF⁶⁵. The best characterized mammalian regulators are SR proteins, a family of splicing factors which contain arginine(R)-serine(S)-rich sequences (reviewed in Fu, X. D. 1995, RNA 1:663; Manley, J. L. and Tacke, R. 1996, Genes Dev 10:1569; Chabot, B. 1996, (supra); Graveley, B. R. 2000, RNA 6:1197). SR proteins generally favor proximal (internal) 5′ splice site or 3′ splice site selection in vitro and exon inclusion in vivo (Fu, X. D. et al. 1992, Proc Natl Acad Sci USA 89:11224; Mayeda, A. et al. 1993, Mol. Cel. Biol. 13:2993; Caceres, J. F. et al. 1994, Science 265:1706; Zahler, A. M. and Roth, M. B. 1995, Proc Natl Acad Sci USA 92:2642). One member of this family of proteins (SF2/ASF) promotes U1 snRNP binding to weak 5′ splice sites, and favors U1 snRNP binding to all 5′ splice sites in more complex pre-mRNAs (Eperon, I. C. et al. 1993, EMBO J. 12:3607). Thus, increasing the proportion of pre-mRNA molecules bound by U1 at all competing 5′ splice sites should favor the use of the internal site because of its closer proximity to the 3′ splice site. In addition, U2AF⁶⁵ binding to weak 3′ splice sites is stimulated by SR proteins bound to a downstream splicing enhancer (e.g., purine-rich sequence) (Lavigueur, A. et al. 1993, Genes Dev. 7:2405; Sun, Q. et al. 1993, Genes Dev. 7:2598; Staknis, D. and Reed, R. 1994, Mol. Cell. Biol. 14:7670; Wang, Z. et al. 1995, RNA 1:21). Because SR proteins can interact simultaneously with U2AF and the U1 snRNP 70K protein (Wu, J. Y. and Maniatis, T., 1993, Cell 75:1061), SR proteins are thought to participate in the stimulation of U2AF binding through exon-bridging interactions with a downstream U1-bound 5′ splice sites (Wang et al., supra). Current results also suggest that SR proteins promote commitment between a pair of splice site by favoring an intron-bridging interaction between U1 snRNP and U2AF (Wu, J. Y. and Maniatis, T. 1993, supra). While SR proteins can stimulate each of the initial ATP-independent steps of spliceosome assembly, in some cases SR proteins may act as splicing repressors, either by binding to sites that sterically occlude spliceosome assembly (Kanopka, A. et al. 1996, Nature 381:535), or by blocking the binding of more active SR proteins (Gallego, M. E. et al. 1997, EMBO J. 16:1772). The current understanding of the role of SR proteins is still rudimentary and more work is needed to understand the biological function of each member, and the role of phosphorylation by specific kinases that modulate their localization and activity in the nucleus (Gui, J. F. et al. 1994, Nature 369:678; Colwill, K. et al. 1996, EMBO J. 15:265; Xiao, S. H. and Manley, J. L. 1997, Genes Dev. 11:334). There thus remains a need to better understand the role of SR proteins and of phosphorylation by specific kinases in alternative splicing. There also remains a need to identify modulators of SR function in splicing and more particularly in alternative splicing.

The negative regulation of U1 snRNP and U2AF⁶⁵ binding is also a strategy used to modulate splice site selection and often requires the participation of hnRNP or related proteins. In the Drosophila, somatic P-element pre-mRNA, the formation of an RNA-protein complex containing U1 snRNP, the soma-specific PSI protein and the ubiquitous hrp48 protein prevents downstream 5′ splice site recognition (Siebel, C. W. et al. 1995, Genes Dev. 9:269). HnRNP I, also called PTB, has been implicated in the regulation of several alternatively spliced genes, including alpha- and beta-tropomyosin, c-src and GABA_{A} receptor gamma 2 subunit, possibly by interfering with the adjacent binding of U2AF (reviewed in Valcarcel, J. and Gebauer, F. 1997, Curr. Biol. 7:R705). The hnRNP F protein has been found to be part of a complex involved in activating neural-specific splicing of the alternative c-src exon N1 (Min, H. et al. 1995, Genes Dev 9:2659). The more abundant members of the family of hnRNP proteins (A and B group) can antagonize the effect of SR proteins on splice site selection (Mayeda, A. and Krainer, A. R. 1992, Cell 68:365; Mayeda, A. et al. 1993, supra; Yang, X. et al. 1994, Proc Natl Acad Sci USA 91:6924).

The heterogeneous nuclear ribonucleoparticle (hnRNP) protein A1 is one of the most abundant nuclear protein in actively growing mammalian cells. The hnRNP A1 pre-mRNA is itself alternatively spliced to yield the A1 and A1b proteins which differ in their ability to affect splice site selection. HnRNP A1 affects 5′ splice site selection through the presence of high-affinity A1 binding sites (Blanchette, M and Chabot, B. 1999, EMBO J. 18:1939).

Dimethyl sulfoxide (DMSO) is often used to promote cell differentiation of tumor cell lines. For example, the treatment of mouse erythroleukemic cells and mouse neuroblastoma cells with 2% DMSO induces morphological changes and phenotypic differentiation into red blood cells and neurons, respectively. DMSO also promotes differentiation of rhabdomyosarcoma cells in vitro (Prados, J. et al. 1993, Cell Mol. Biol. 39:525), induces the differentiation and apoptosis of the human U937 monoblast leukemia cell line into monocyte/macrophage (Nicholson et al. 1992, J. Biol. Chem. 267:17849; Chateau et al. 1996, Anal. Cell Pathol. 10:75), and stimulates the differentiation of a human ovarian adenocarcinoma cell line (Grunt et al. 1992, J. Cell. Sci. 103:501). DMSO is also used to promote the differentiation of hepatocytes in culture (Kojima et al. 1997, Hepatology 26:585). Contrastingly, DMSO can be used to prevent terminal differentiation of myoblasts (Blau and Epstein 1979, Cell 17:95), to inhibit adipocytes differentiation (Wang and Scott 1993, Cell Prolif. 26:55), to block the differentiation of antibody-producing plasma cells (Teraoka et al. 1996, Exp. Cell Res. 222:218), and to interfere with the differentiation of chick embryo chondrocytes (Manduca et al. 1988, Dev. Biol. 125:234). More recently, DMSO treatment has been used to either induce apoptosis (or programmed cell-death) in a pre-T cell line (Trubiani et al. 1999, Cell Prolif. 32:119), or, in contrast, to inhibit cell density-dependent apoptosis (Fiore and Degrassi 1999, Exp. Cell Res. 251:102). Thus, depending on the cell line used, DMSO can either promote differentiation and apoptosis, or block differentiation and apoptosis. The use of DMSO in pharmaceutical formulations is known, for example, from U.S. Pat. Nos. 4,296,104; 4,652,557; and 5,516,526.

The mechanisms by which these events occurs are unclear however. Because DMSO is used to facilitate DNA uptake during transfection procedures (e.g., Melkonyan et al. 1996, Nucl. Acids. Res. 24:4356), it has been proposed to affect cell membrane and signal transduction. Consistent with this view, DMSO treatment can affect the expression of protein kinase C (Makowske et al. 1988, J. Biol. Chem. 263:3402). DMSO treatment has been shown to also promote changes in the abundance of certain mRNAs and spliced isoforms (Tam et al. 1997, J. Lipid Res 38:2090; Srinivas et al. 1991, Exp. Cell Res. 196:279; Bahler and Lord 1985, J. Immunol. 134:2790; Campbell et al. 1990, Genes Dev. 4:1252). It is not clear yet how DMSO modulates the level of mRNAs. It still remains to be determined whether this effect is direct or indirect. It also remains to be determined whether transcription, and/or signal transduction (a kinase or phosphatase) and/or splicing is or are involved or responsible for this modulation. Among the genes reported to be affected in their alternative splicing is the NCAM pre-mRNA. A 2% DMSO treatment of N2a cells promotes an increase in the inclusion of neuro-specific exon 18 (Pollerberg et al. 1985, J. Cell. Biol. 101: 1921; Prentice et al. 1987, EMBO J. 6: 1859). Genes whose alternative splicing profiles have been reported to be affected by treatment with DMSO include the amyloid precursor protein (Pan et al. 1993, Brain Res Mol Brain Res. 18:259), the serotonin 5-HT3 receptor-A mRNA (Emerit et al. 1995, J. Neurochem. 65:1917), p53 (Bendori, 1987. Virology 161:607). DMSO has also been associated to affect c-myc mRNA elongation and maturation (Eick 1990, Nucl. Acids Res. 18:1199) and the mRNA translation of other genes (Yenofsky et al., 1983, Mol. Cell. Biol. 3:1197). A survey of the scientific literature reveals that the mechanism of action of DMSO, as far as gene expression is concerned, has not been throroughly investigated. There thus remains a need to identify the mechanism of action of DMSO in gene expression.

The present invention seeks to meet these and other needs.

The present description refers to a number of documents, the content of which is herein incorporated by reference in their entirety.

### SUMMARY OF THE INVENTION

It is therefore an aim of the present invention to overcome the drawbacks in the prior art and enable a better understanding of the relevance of alternative splicing to cell growth, cell differentiation and mammalian development.

The present invention also relates to a better understanding of how splicing and the regulation thereof are controlled in cells and provides agents which can modulate splicing and/or alternative splicing in cells and animals.

In one particular embodiment, the present invention seeks to provide agents which can modulate alternative splicing through a modulation of SR proteins function. In a preferred embodiment, such agents are polar aprotic solvents. In an especially preferred embodiment of the present invention, DMSO, DMF, formamide and related compounds are used to control alternative splicing by modulation of SR protein-dependent alternative splicing.

The present invention also relates to a normalization of splicing and/or alternative splicing activity in splicing extracts (e.g. S100 and more particularly nuclear extracts) using SR function modulating agents such as DMSO, DMF, formamide and related compounds.

The present invention also provides methods and assays to identify agents which can modulate alternative splicing. In one particular embodiment of the present invention, such a method comprises an incubation with a splicing extract which contains pre-mRNA and a compound which modulates the alternative splicing activity of SR proteins, wherein a change in splicing of this pre-mRNA can be detected and/or measured, and comparing qualitatively or quantitatively the splicing of this pre-mRNA in the presence of an agent (or a library thereof) and in the absence thereof wherein an agent which can modulate splicing is identified when a qualitative or quantitative difference in the spliced products or splicing intermediates is measurably different in the presence of the agent as compared to in the absence thereof. In a particularly preferred embodiment, the compound which modulates SR activity is DMSO, DMF, formamide or related compound.

The present invention also provides a method of modulating SR protein functions in alternative splicing, comprising a treatment thereof into an alternative-splicing modulating amount of an agent selected from the group consisting of DMSO, DMF, formamide and related compounds.

In addition, the invention relates to a method of modulating the ratio of alternatively spliced isoforms relative to each other, as well as to normalize the alternative splicing activity of a splicing extract, which comprises an incubation of a cell or extract with an alternative splicing modulating amount of a polar aprotic solvent such as DMSO, DMF, formamide or related compounds.

In another embodiment, the present invention enables an identification of new alternatively spliced units comprising an incubation of a cell or splicing extract with an alternative splicing modulating amount of a polar aprotic solvent such as DMSO, DMF, formamide and the like, whereby an alternative splicing modulating amount of DMSO, DMF, formamide or related compound through their normalizing activity can enable the detection and identification of previously unrecognized alternatively spliced units.

In yet another embodiment, the present invention provides alternative splicing kits, comprising an agent of the present invention which modulates the splicing and/or alternative splicing activity of SR proteins, as well as splicing reagents.

It is shown herein that DMSO can control alternative RNA splicing directly. This direct link is based on the demonstration that DMSO affects the alternative splicing of pre-mRNAs when assayed in extracts prepared from human HeLa cells (i.e., an in vitro splicing system).

Thus, the effects observed must affect factors involved in alternative splicing because the effects seen cannot be occurring through membrane-mediated events, transcription, translation, etc. It thus demonstrates that the effect of DMSO or the like on splicing is sufficient to modulate alternative splicing unit selection.

DMSO was shown not to affect constitutive (generic) splicing in nuclear extracts. A pre-mRNA which has been used as a model to study constitutive splicing remains spliced efficiently in the presence of up to 2.5% DMSO. However, at the same concentration, DMSO can completely abrogate distal 5′ splice site utilization when using a model pre-mRNA that has been used previously to show that the binding of hnRNP A1 to high affinity A1 binding sites can promote distal 5′ splice site selection (Blanchette and Chabot 1999, EMBO J. 18: 1939). When distal 5′ splice site selection is promoted by an hnRNP A1 independent mechanism (e.g., secondary structure formation), 5′ splice site selection is not affected by DMSO. DMSO does not affect the activity of hnRNP A1 since the addition of recombinant A1 protein in the DMSO-containing extract can shift splice site selection to the distal 5′ splice site as efficiently as in an extract lacking DMSO. This effect is also specific since the addition of equivalent amounts of GST or gene 32 protein has no effect when added to DMSO-containing extracts. In addition, DMSO affects generic splicing in S-100 extracts which are post-nuclear extracts containing residual amounts of SR proteins and U2AF proteins.

DMSO also affects 3′ splice site selection. Using model pre-mRNAs that are efficiently spliced to the distal 3′ splice site in a SR-dependent manner, DMSO was shown herein to enable a shift in the proportion of proximal/distal 3′ splice site use.

It is shown that DMSO affects NCAM splice site selection in vitro using a model pre-mRNA carrying a NCAM 3′ splice site. It was already known that the treatment of N2a cells with DMSO promoted inclusion of NCAM exon 18. These results strongly suggest that DMSO affects cell differentiation through a direct modulation of alternative splicing.

While the effect of DMSO and related compounds on splicing is exemplified herein with three model pre-mRNAs, the instant invention should not be so limited. Indeed, the present invention provides among other things (1) the means to modulate alternative splicing and/or generic splicing of pre-mRNAs in general; (2) methods of identifying new alternatively spliced units (the joining of novel splice sites, which generate a new splicing unit), and (3) the means of identifying agents which modulate splicing and/or alternative splicing.

DMSO (and related compounds) is shown here to control the alternative splicing profile of pre-mRNAs when SR proteins are involved in the modulation of splicing. Since the understanding of the mechanism of alternative splicing is still limited, few pre-mRNAs have been shown to be dependent on SR proteins to control their alternative splicing profile. Of note, however, when formally tested, one or several SR proteins have been generally, if not always, shown to be implicated in the modulation of splice site selection of a particular pre-mRNA. Thus, it is expected that the present invention will be applicable to numerous mRNA precursors.

The potential of alternative splicing as a cornerstone pathway which could provide the required complexity, tissue specificity, and function for the 30,000 or so genes identified in humans should not be underestimated. In certain conditions, it may be suitable to modulate the alternative splicing profile of a pre-mRNA in order to generate protein isoforms with different activities. Depending on the target pre-mRNA and the type of cells where this gene is expressed, modulating alternative splicing may have a considerable impact on cell growth, other cell properties and homeostasy. For example, certain types of cancer may produce protein isoforms that are important for continued cell growth. Thus, the use of an agent which could modify the alternative splicing profile of a pre-mRNA could give rise to at least one different (or changing the level of) protein isoform which could have a major impact on cell growth and the control thereof, to take but one example. Of course this agent could also quantitatively affect the ratio of alternatively spliced units, thereby modulating the metabolic profile of a cell. In a particular embodiment, DMSO treatment may shift the profile of alternative splicing and promote the production of a protein isoform that prevents cell growth or promotes cell death. In other conditions affecting another cell type expressing different subsets of genes, the reverse may be true, i.e., DMSO may abrogate apoptosis or permit cell division. The identification of the effect of DMSO and related compounds will enable an identification of new genes in which alternative splicing occurs. While such information is already available for a limited number of genes, more will be available soon thanks to the DNA chips technology (e.g. microarrays), other procedures and the teachings of the present invention.

Thus, the polar aprotic solvents of the present invention such as DMSO could be used to treat a variety of conditions including cancer and any disease or condition wherein the expression of certain spliced isoforms makes an important contribution to the disease or condition (e.g. physiologically relevant). In such a case a topical application of DMSO may shift the splicing profile to correct a defect or condition. For example, DMSO might promote cell death by altering the alternative splicing profile of a target pre-mRNA that encodes for example a transcription control factor, a membrane receptor, and other cell growth modulators.

In one particular embodiment of the present invention, a variety of polar aprotic solvent such as DMSO, DMF and formamide derivatives can be produced chemically and tested in an assay of the present invention. These may be more potent than DMSO to modulate alternative splicing. Although DMSO can be viewed as a relatively safe product, derivatives may have additional advantages when topical application is considered.

While in accordance with one embodiment of the present invention, DMSO and related compounds are shown to modulate splicing in extracts, it should be recognized that other agents or compounds could have similar effects. The present invention provides the means to identify such splicing modulators. A non-limiting example of such assay is a screening of agents for identifying denormalizing agents, which revert the normalizing effect of DMSO on alternative splicing site selection.

In accordance with the present invention there is therefore provided a method to modulate splicing and/or alternative splicing in vitro comprising an administration to a cell or extract thereof of an effective amount of a polar aprotic solvent, whereby the effective amount modulates splicing and/or alternative splicing as compared to an untreated cell or extract.

In accordance with the present invention the is also provided a method of modulating the splicing and/or alternative splicing activity of a SR protein comprising: an administration to a cell or extract thereof containing a SR protein of an effective amount of a polar aprotic solvent, whereby the effective amount modulates the activity of the SR protein as compared to a non-treated cell or extract.

In addition, in accordance with the present invention there is provided a splicing kit comprising: a) a container containing a splicing and/or alternative splicing-competent extract; b) a second container containing a splicing and/or alternative splicing buffer; and c) a polar aprotic solvent.

In accordance with the present invention there is also provided a method to normalize a splicing and/or alternative splicing activity of an extract comprising an addition thereto of an effective amount of a polar aprotic solvent, whereby the effective amount normalizes splicing and/or alternative splicing as compared to an untreated extract.

As used herein, the terms “molecule”, “compound”, “agent” or “ligand” are used interchangeably and broadly to refer to natural, synthetic or semi-synthetic molecules or compounds. The term “molecule” therefore denotes for example chemicals, macromolecules, cell or tissue extracts (from plants or animals) and the like. Non limiting examples of molecules include nucleic acid molecules, peptides, ligands (including, for example, antibodies and carbohydrates) and pharmaceutical agents. The agents can be selected and screened by a variety of means including random screening, rational selection and by rational design using for example SR protein or modelling methods such as computer modelling. As will be understood by the person of ordinary skill, macromolecules having non-naturally occurring modifications are also within the scope of the term “molecule”. As will also be understood by a person of ordinary skill, various assays can be used to identify such compounds. Non-limiting examples thereof include splicing assays and binding assays. For example, peptidomimetics, well known in the pharmaceutical industry and generally referred to as peptide analogs can be generated by modelling as mentioned above. The molecules identified in accordance with the teachings of the present invention have a therapeutic value in diseases or conditions in which splicing and more particularly alternative splicing or modulated in a fashion which affects cellular homeostasy. Alternatively, the molecules identified in accordance with the teachings of the present invention find utility in the development of compounds which can modulate the activity of SR proteins (or other splicing factors) in splicing and/or from splicing.

Generally, high throughput screens for one or more SR protein splicing modulators i.e. candidate or test compounds or agents (e.g., peptides, peptidomimetics, small molecules or other drugs) may be based on assays which measure biological activity of SR proteins. The invention therefore provides a method (also referred to herein as a “screening assay”) for identifying modulators, which have a stimulatory or inhibitory effect on, for example, SR protein biological activity, or which bind to or interact with SR proteins, or which have a stimulatory or inhibitory effect on, for example, the expression or activity of SR interacting proteins (targets) or substrates.

In one embodiment, an assay is a cell-based assay in which a cell which expresses a SR protein or biologically active portion thereof, either natural or recombinant in origin, is contacted with a test compound and the ability of the test compound to modulate SR biological activity, e.g., modulation of alternative splicing activity, or of splicing, or any other measurable biological activity of SR is determined. Determining the ability of the test compound to modulate SR activity can be accomplished by monitoring, for example, the spliced RNA unit or of the protein encoded thereby upon exposure of the test compound to the cell. DMSO or related compounds could be used as controls. Furthermore, determining the ability of the test compound to modulate SR activity can be accomplished by preparing a splicing extract from the cell treated or not with a compound and comparing the alternative splicing activity thereof.

In yet another embodiment, an assay of the present invention is a cell-free assay in which a SR protein or biologically active portion thereof, either naturally occurring or recombinant in origin, is contacted with a test compound and the ability of the test compound to bind to, or otherwise modulate the biological activity of the SR protein or biologically active portion thereof is determined.

In another embodiment, the assay is a cell-free assay in which a SR protein or biologically active portion thereof is contacted with a test compound and the ability of the test compound to modulate (e.g., stimulate or inhibit) the activity of the SR protein or biologically active portion thereof is determined. Determining the ability of the test compound to modulate the activity of a SR protein can be accomplished, for example, by determining the ability of the SR protein to bind to a SR target molecule by one of the methods described above for determining direct binding. Such protein interaction determination can be accomplished using a technology such as real-time Biomolecular Interaction Analysis (BIA, Sjolander, S. and Urbaniczky, C. (1991) Anal. Chem. 63:2338-2345 and Szabo et al. (1995) Curr. Opin. Struct. Biol. 5:699-705). As used herein, “BIA” refers to a technology for studying biospecific interactions in real time, without labeling any of the interactants (e.g. BIA core). Changes in the optical phenomenon of surface plasmon resonance (SPR) can be used as an indication of real-time reactions between biological molecules.

Of course, and as exemplified herein, the determination of the test compound to modulate SR protein activity can be determined by assessing the splicing profile of a treated versus non-treated splicing extract.

The assays described above may be used as initial or primary screens to detect promising lead compounds for further development. Often, lead compounds will be further assessed in additional, different screens. Therefore, this invention also includes secondary SR protein screens.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, Anticancer Drug Des. 12: 145, 1997). Examples of methods for the synthesis of molecular libraries can be found in the art, for example in: DeWitt et al. (1993) Proc. Natl. Acad. Sci. USA. 90:6909; Erb et al. (1994) Proc. Natl. Acad. Sci. USA 91:11422; Zuckermann et al. (1994), J. Med. Chem. 37:2678; Cho et al. (1993) Science 261:1303; Carrell et al. (1994) Angew. Chem, Int. Ed Engl. 33:2059; ibid, Angew. Chem. Jnl. Ed. Engl. 33:2061; and in Gallop et al. (1994). Med Chem. 37:1233. Libraries of compounds may be presented in solution (e.g. Houghten (1992) Biotechniques 13:412-421) or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990); Science 249:386-390). Examples of methods for the synthesis of molecular libraries can be found in the art, for example in: DeWitt et al. (1993) Proc. Natl. Acad. Sci. USA. 90:6909; Erb et al. (1994) Proc. Natl. Acad. Sci. USA 91: 11422; Zuckermann et al. (1994), J: Med. Chem. 37:2678; Cho et al. (1993), Science 261:1303; Carrell et al. (1994) Angew. Chem Int. Ed. Engl. 33:2059, or luciferase, and the enzymatic label detected by determination of conversion of an appropriate substrate to product.

In summary, based on the disclosure herein, those skilled in the art can develop splicing modulator screening assays and more particularly SR protein-dependent splicing modulator screening assays. The assays of this invention may be developed for low-throughput, high-throughput, or ultra-high throughput screening formats.

For the purpose of the present invention, the following abbreviations and terms are defined below.

The terminology “SR” relates to splicing regulating protein rich in serines (S) and arginines (R) (hence SR proteins) having an activity in splicing and alternative splicing. It will be clear to the skilled artisan that recombinants, derivatives or portions of SR splicing factors can also be used and tested in accordance with the present invention. SR proteins have been described for example in Chabot 1996 (supra) and Graveley 2000 (supra).

The terminology “SR protein biological activity” or the like refers to the activity of SR proteins in splicing and/or alternative splicing. These activities can be tested in accordance with the methods and assays of the present invention as well as other methods and assays in the art (e.g. splicing assays, alternative splicing assays, spliceosone formation, binding assays, protein-protein interaction assays between SR and another factor involved in splicing, gel shift assays, etc.). Blanchette and Chabot 1999 (supra) provides examples of some of these assays and is hereby incorporated by reference. Of course, since a number of SR proteins have been characterized, recombinant SR proteins could be used (as exemplified hereinbelow).

The terminology “a splicing and/or alternative splicing modulating amount of a compound” or a similar terminology refers to an amount which shows a detectable and significant modulation of splicing and/or alternative splicing in the presence of the compound as compared to in the absence thereof. This amount can be adapted to particular extracts or to particular conditions of use (cell or extract treatment) by a person of ordinary skill in accordance with the teachings of the present invention.

As used herein “normalizing”, “normalized” and the like refer to a standardization effected by a polar aprotic solvent of the present invention on splicing and/or alternative splicing. As exemplified herein, the solvents of the present invention can enable a standardization of the extracts, such that a more homogeneous splicing and/or alternative splicing activity is found between different batches of extracts (hence normalized extracts).

The present invention also features a pharmaceutical composition which includes an alternative splicing and/or splicing modulating amount of an agent selected from DMSO, DMF, formamide and related compounds.

It is notable that administration of modulators of SR function in alternative splicing is not expected to be detrimental to any particular individual or animal. Of note, DMSO is already used for the treatment or prevention of certain diseases or conditions. A person of ordinary skill should adapt the doses and regimen in order to avoid deleterious effects in animals, patients or cells.

In addition, the term “therapeutically effective amount” of an inhibitor or modulator is a well-recognized phrase. The amount actually applied will be dependent upon the individual or animal to which treatment is to be applied, and will preferably be an optimized amount such that an inhibitory effect is achieved without significant side-effects (to the extent that those can be avoided by use of the inhibitor). That is, if effective inhibition can be achieved with no side-effects with the inhibitor at a certain concentration, that concentration should be used as opposed to a higher concentration at which side-effects may become evident. If side-effects are unavoidable, however, the minimum amount of inhibitor that is necessary to achieve the inhibition desired should be used. The terminology “effective amount” should be similarly understood while, in certain embodiments, the aspect of side effects and the like may or may not come into consideration. Of course, the present invention provides means to determine the splicing effective amounts.

By “inhibitor” is simply meant any reagent, drug or chemical which is able to inhibit the alternative splicing activity of SR proteins in vivo or in vitro. Such inhibitors can be readily identified using standard screening protocols in which an SR protein placed in contact with a potential inhibitor and the level of splicing or the spliced products are measured or identified in the presence or absence of the inhibitor or in the presence of varying amounts thereof. In this way, not only can useful inhibitors (or stimulators) be identified, but the optimum level of such an inhibitor (or stimulator) can be determined in vitro. Once identified as a modulator in vitro, the agent can be tested in vivo. Numerous methods to test the in vivo effect of this modulator are known to the person skilled in the art to which this application pertains. In one particular embodiment, this agent is DMSO or derivatives thereof, DMF or derivatives thereof, and formamide and derivatives thereof.

DMSO is a known chemical (see, for example, the Merck Index, 11th Edition, pages 513-514 and references therein). DMSO derivatives will be understood by a person skilled in the art to be an equivalent of C₂H₆OS, which retain their function as modulator of splicing and particularly of alternative splicing.

The terminology “DMSO derivatives” or the like should be understood to refer to polar aprotic solvents. Polar aprotic solvents (such as for example DMSO, DMF, formamide) have relatively high dipole moments due to polar bonds and do not have H atoms that can be donated into an H-bond. DMSO has a dipole moment of 3.96, DMF has a dipole moment of 3.79 and formamide has a dipole moment of 3.37. Solvent polarity is usually expressed in terms of dielectric constants, which measures the ability of a solvent to act as an insulator of electric charges. DMSO has a dielectric constant of 47, DMF has a dielectric constant of 38 whereas formamide has a dielectric constant of 111. Other non-limiting examples of solvents which could be used in accordance with the present invention include HMPA, N-methylformamide, nitromethane, acetone, and acetonitrile.

As used herein, the terms “molecule”, “compound” or “ligand” are used interchangeably and broadly to refer to natural, synthetic or semi-synthetic molecules or compounds. The term “molecule” therefore denotes for example chemicals, macromolecules, cell or tissue extracts (from plants or animals) and the like. The agents can be selected and screened by a variety of means including random screening, rational selection and by rational design using for example protein or ligand modeling methods such as computer modeling, combinatorial library screening and the like. The terms “rationally selected” or “rationally designed” are meant to define compounds which have been chosen based on the configuration of the interaction domains of the present invention. As will be understood by the person of ordinary skill, macromolecules having non-naturally occurring modifications are also within the scope of the term “molecule”. For example, peptidomimetics, well known in the pharmaceutical industry and generally referred to as peptide analogs can be generated by modeling as mentioned above. Such molecules or compounds can be screened using an assay in accordance with the present invention in view of identifying and/or characterizing splicing modulating agents or molecules. The molecules identified in accordance with the teachings of the present invention have a therapeutic value in diseases or conditions in which the physiology or homeostasis of the cell and/or tissue is compromised by a defect in splicing and in splicing and especially in alternative splicing.

As used herein, agonists and antagonists of the SR-dependent splicing activity also include potentiators of known compounds with such agonist or antagonist properties. In one embodiment, agonists can be detected by contacting the indicator cell with a compound or mixture thereof or library of molecules (e.g. combinatorial library) for a fixed period time and determining a biological activity as described herein. Of course, antagonists can be similarly detected.

The agents identified in accordance with an assay of the present invention are likely to find utility for modulating numerous types of metabolic pathways in animals, tissues, cells, and extracts, and are likely to have an effect on one or more of pathways, diseases or conditions. In view of the ubiquitous nature of alternative splicing in higher eukaryotic cells and in particular in mammals, the instant invention provides the means to identify and characterize alternative splicing events and agents which modulate same in a variety (if not most, or all) of diseases or conditions. Non limiting examples of such pathways and agents which modulate same include proliferation, differentiation, apoptosis, inflammatory response, neoplasia, immune response, senescence, memory and neuronal activity, muscle contraction, tissue regeneration, obesity, anemia, diabetes, hypertension, Alzheimer, signal transduction, membrane potential, transcription, translation, transport, protein shuttling, secretion.

Broadly, therefore, the present invention enables a modification of the splicing pattern in a cell and in so doing of a modification, or of a modulation of one or more pathways therein, through a splicing pattern-modulating amount of DMSO, DMF, formamide or the like. When applied to in vitro systems the present invention enables the identification of new splicing units in genes which can then be more formally tested in in vitro and/or in vivo for their physiological relevance in cell metabolism or homeostasy.

Unless defined otherwise, the scientific and technological terms and nomenclature used herein have the same meaning as commonly understood by a person of ordinary skill to which this invention pertains. Generally, the procedures for cell cultures, infection, molecular biology methods and the like are common methods used in the art. Such standard techniques can be found in reference manuals such as for example Sambrook et al. (1989, Molecular Cloning—A Laboratory Manual, Cold Spring Harbor Laboratories) and Ausubel et al. (1994, Current Protocols in Molecular Biology, Wiley, New York).

The present description refers to a number of routinely used recombinant DNA (rDNA) technology terms. Nevertheless, definitions of selected examples of such rDNA terms are provided for clarity and consistency.

As used herein, “nucleic acid molecule”, refers to a polymer of nucleotides. Non-limiting examples thereof include DNA (i.e. genomic DNA, cDNA) and RNA molecules (i.e. pre-mRNA). The nucleic acid molecule can be obtained by cloning techniques or synthesized. DNA can be double-stranded or single-stranded (coding strand or non-coding strand [antisense]).

The term “recombinant DNA” as known in the art refers to a DNA molecule resulting from the joining of DNA segments. This is often referred to as genetic engineering.

The terms “DNA segment” or “RNA segment” refer to a DNA molecule or RNA molecule comprising a linear stretch or sequence of nucleotides. This sequence when read in accordance with the genetic code, can encode a linear stretch or sequence of amino acids which can be referred to as a polypeptide, protein, protein fragment and the like. As known in the art, through splicing, RNA segments are joined together, leaving intervening sequences behind. Through alternative splicing, the selection and joining of particular RNA segments give rise to structurally different and often functionally different polypeptides.

The nucleic acid (i.e. DNA, RNA or pre-mRNA) for practicing the present invention may be obtained according to well-known methods.

Oligonucleotide probes or primers of the present invention may be of any suitable length, depending on the particular assay format and the particular needs and targeted sequences employed. In general, the oligonucleotide probes or primers are at least 12 nucleotides in length, preferably between 15 and 24 molecules, and they may be adapted to be especially suited to a chosen nucleic acid amplification system. As commonly known in the art, the oligonucleotide probes and primers can be designed by taking into consideration the melting point of hybridization thereof with its targeted sequence (see below and in Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

“Nucleic acid hybridization” refers generally to the hybridization of two single-stranded nucleic acid molecules having complementary base sequences, which under appropriate conditions will form a thermodynamically favored double-stranded structure. Examples of hybridization conditions can be found in the two laboratory manuals referred above (Sambrook et al., 1989, supra and Ausubel et al., 1989, supra) and are commonly known in the art. In the case of a hybridization to a nitrocellulose filter, as for example in the well known Southern blotting procedure, a nitrocellulose filter can be incubated overnight at 65° C. with a labeled probe in a solution containing 50% formamide, high salt (5×SSC or 5×SSPE), 5× Denhardt's solution, 1% SDS, and 100 μg/ml denatured carrier DNA (i.e. salmon sperm DNA). The non-specifically binding probe can then be washed off the filter by several washes in 0.2×SSC/0.1% SDS at a temperature which is selected in view of the desired stringency: room temperature (low stringency), 42° C. (moderate stringency) or 65° C. (high stringency). The selected temperature is based on the melting temperature (Tm) of the DNA hybrid. Of course, RNA-DNA hybrids can also be formed and detected. In such cases, the conditions of hybridization and washing can be adapted according to well-known methods by the person of ordinary skill. Stringent conditions will be preferably used (Sambrook et al., 1989, supra).

Probes of the invention can be utilized with naturally occurring sugar-phosphate backbones as well as modified backbones including phosphorothioates, dithionates, alkyl phosphonates and a-nucleotides and the like. Modified sugar-phosphate backbones are generally taught by Miller, 1988, Ann. Reports Med. Chem. 23:295 and Moran et al., 1987, Nucleic Acids Res., 14:5019. Probes of the invention can be constructed of either ribonucleic acid (RNA) or deoxyribonucleic acid (DNA).

The types of detection methods in which probes can be used include Southern blots (DNA detection), dot or slot blots (DNA, RNA), and Northern blots (RNA detection). Although less preferred, labeled proteins could also be used to detect a particular nucleic acid sequence to which it binds. Other detection methods include kits containing probes on a dipstick setup and the like.

Although the present invention is not specifically dependent on the use of a label for the detection of a particular nucleic acid sequence, such a label might be beneficial, by increasing the sensitivity of the detection. Furthermore, it enables automation. Probes can be labeled according to numerous well-known methods (Sambrook et al., 1989, supra). Non-limiting examples of labels include ³H, ¹⁴C, ³²P, and ³⁵S. Non-limiting examples of detectable markers include ligands, fluorophores, chemiluminescent agents, enzymes, and antibodies. Other detectable markers for use with probes, which can enable an increase in sensitivity of the method of the invention, include biotin and radionucleotides. It will become evident to the person of ordinary skill that the choice of a particular label dictates the manner in which it is bound to the probe.

As commonly known, radioactive nucleotides can be incorporated into nucleic acids or probes of the invention by several methods. Non-limiting examples thereof include kinasing the 5′ ends of the probes using gamma ³²P ATP and polynucleotide kinase, using the Klenow fragment of Pol I of E. coli in the presence of radioactive dNTP (i.e. uniformly labeled DNA probe using random oligonucleotide primers in low-melt gels), using the SP6/T7 system to transcribe a DNA segment in the presence of one or more radioactive NTP, and the like.

As used herein, “oligonucleotides” or “oligos” define a molecule having two or more nucleotides (ribo or deoxyribonucleotides). The size of the oligo will be dictated by the particular situation and ultimately on the particular use thereof and adapted accordingly by the person of ordinary skill. An oligonucleotide can be synthetised chemically or derived by cloning according to well-known methods.

As used herein, a “primer” defines an oligonucleotide which is capable of annealing to a target sequence, thereby creating a double stranded region which can serve as an initiation point for DNA synthesis under suitable conditions.

Amplification of a selected, or target, nucleic acid sequence may be carried out by a number of suitable methods. See generally Kwoh et al., 1990, Am. Biotechnol. Lab. 8:14-25. Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the Qbetaβ replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra). Preferably, amplification will be carried out using PCR.

Polymerase chain reaction (PCR) is carried out in accordance with known techniques. See, e.g., U.S. Pat. Nos. 4,683,195; 4,683,202; 4,800,159; and 4,965,188 (the disclosures of all three U.S. patent are incorporated herein by reference).

As used herein, the term “gene” is well known in the art and relates to a nucleic acid sequence which traditionally has been recognized as defining a single protein or polypeptide. Of course, alternative splicing enables the production of more than one polypeptide from a single gene. A “structural gene” defines a DNA sequence which is transcribed into RNA and translated into a protein having a specific amino acid sequence thereby giving rise to a specific polypeptide or protein. It will be readily recognized by the person of ordinary skill, that DMSO and related compounds of the present invention can be incorporated into splicing kit formats which are well known in the art, to modulate splicing and/or alternative splicing in the extract. In one embodiment, different amounts of DMSO could be added to the extract and tested on a chosen pre-mRNA to identify and/or validate new splicing units. The person of ordinary skill will understand that the characterization of the splicing units can be carried-out by a number of conventional molecular biology methods. It will be recognized by the person of ordinary skill that DMSO and related compounds can be incorporated into any one of numerous established kits formats which are well known in the art, in order to improve and/or modulate splicing.

The present invention therefore also relates to a splicing kit which comprises DMSO or related compound in accordance with the present invention. For example, a compartmentalized kit in accordance with the present invention includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers will include a container which will accept the test sample (e.g. a chosen pre-mRNA), a container which contains an extract used in the assay, a container which contains the SR activity modulating compound of the present invention (e.g. DMSO), and containers which contain reagents to enable splicing to occur or to enable detection of the spliced units or intermediates. Of course, the extract used in the assay could have been mixed with the SR activity modulating compound of the present invention (e.g. DMSO).

In a particular embodiment, the kit would comprise a container containing a nuclear extract whose alternative splicing behavior has been normalized by the addition of DMSO, a container which can accept one or more pre-mRNA, one or more containers containing the reagents enabling splicing to occur and instructions as to how to perform the in vitro splicing experiments using the kit. In one embodiment, the nuclear extract is a transcription extract.

It should be understood that the splicing-competent extracts (which also include alternative splicing-competent extracts) of the present invention can be prepared by a number of protocols well known in the art. Furthermore, such extracts can be prepared from numerous types of cells or cell lines as commonly known. In accordance with one aspect of the present invention, a cell or cell line can be chosen so as to analyze tissue or cell-specific splicing and especially alternative splicing modulation and to identify splicing units and the regulation of their expression.

As exemplified herein, the cells can also be treated directly with the splicing modulating agent. Numerous types of cells are amenable to such in vivo treatments. Since agents such as DMSO have been used in vivo in animals, the present invention also provides for in vivo treatment of animals with agents of the present invention to modulate splicing and/or alternative splicing.

A “heterologous” (i.e. a heterologous gene) region of a DNA molecule is a subsegment seg/ment of DNA within a larger segment that is not found in association therewith in nature. The term “heterologous” can be similarly used to define two polypeptidic segments not joined together in nature. Non-limiting examples of heterologous genes include reporter genes such as luciferase, chloramphenicol acetyl transferase, β-galactosidase, and the like which can be juxtaposed or joined to heterologous control regions or to heterologous polypeptides.

The term “vector” is commonly known in the art and defines a plasmid DNA, phage DNA, viral DNA and the like, which can serve as a DNA vehicle into which DNA of the present invention can be cloned and for which a RNA of the present invention can be obtained. Numerous types of vectors exist and are well known in the art.

The term “expression” defines the process by which a gene is transcribed into mRNA (transcription), the mRNA is then being translated (translation) into one polypeptide (or protein) or more.

The terminology “expression vector” defines a vector or vehicle as described above but designed to enable the expression of an inserted sequence following transformation into a host. The cloned gene (inserted sequence) is usually placed under the control of control element sequences such as promoter sequences. The placing of a cloned gene under such control sequences is often referred to as being operably linked to control elements or sequences.

Operably linked sequences may also include two segments that are transcribed onto the same RNA transcript. Thus, two sequences, such as a promoter and a “reporter sequence” are operably linked if transcription commencing in the promoter will produce an RNA transcript of the reporter sequence. In order to be “operably linked” it is not necessary that two sequences be immediately adjacent to one another.

Expression control sequences will vary depending on whether the vector is designed to express the operably linked gene in a prokaryotic or eukaryotic host or both (shuttle vectors) and can additionally contain transcriptional elements such as enhancer elements, termination sequences, tissue-specificity elements, and/or translational initiation and termination sites.

Prokaryotic expressions are useful for the preparation of large quantities of the protein or for producing a transcript encoded by the DNA sequence of interest. When used to produce a protein, it can be purified according to standard protocols that take advantage of the intrinsic properties thereof, such as size and charge (i.e. SDS gel electrophoresis, gel filtration, centrifugation, ion exchange chromatography . . . ). In addition, the protein of interest can be purified via affinity chromatography using polyclonal or monoclonal antibodies. The purified protein may be used for therapeutic applications or tested to verify or identify whether an identified protein isoform possesses a quantatively or qualitatively novel property.

As used herein, “chemical derivatives” is meant to cover additional chemical moieties not normally part of the subject matter of the invention. Such moieties could affect the physico-chemical characteristic of the derivative (i.e. solubility, absorption, half life and the like, decrease of toxicity). Such moieties are exemplified in Remington's Pharmaceutical Sciences (1980). Methods of coupling these chemical-physical moieties to a polypeptide are well known in the art.

As used herein, the term “purified” refers to a molecule having been separated from a cellular component. Thus, for example, a “purified protein” has been purified to a level not found in nature. A “substantially pure” molecule is a molecule that is lacking in substantially all other cellular components.

For administration to animals and in particular humans, the prescribing medical professional will ultimately determine the appropriate form and dosage of DMSO and related compounds for a given patient, and this can be expected to vary according to the chosen therapeutic regimen, the response and condition of the patient as well as the severity of the disease.

Composition within the scope of the present invention should contain the active agent in an amount effective to achieve the desired therapeutic effect while avoiding adverse side effects. Pharmaceutically acceptable preparations and salts of the active agent are within the scope of the present invention and are well known in the art (Remington's Pharmaceutical Science, 16th Ed., Mack Ed.). The dosage will be adapted by the clinician in accordance with conventional factors such as the extent of the disease and different parameters from the patient. Since some of the agents of the present invention (e.g. DMSO) have been used in pharmaceutical compositions, the dosage thereof in accordance with the present invention will be adaptable to meet the particular needs of a person of ordinary skill. Compositions comprising up to 85% of DMSO by weight of the solution (for topical administration) have been described (U.S. Pat. Nos. 4,652,257 and 5,516,526).

Other objects features and advantages of the present invention will become apparent upon reading of the following non-restrictive description of the preferred embodiments thereof given by way of example only with reference to the accompanying drawings and from the claims.

### BRIEF DESCRIPTION OF THE DRAWINGS

Having thus generally described the invention, reference will now be made to the accompanying drawings, showing by way of illustration a preferred embodiment thereof, and in which:

FIG. 1 shows that DMSO affects 5′ splice site selection in a cell-free extract. A, structure of the pre-mRNAs used to assay modulation of 5′ splice site selection. C5′ −/− and C5′ 4/4 have been described in Blanchette, M. and Chabot, B. 1999, (supra). The C5′ 4/4 pre-mRNA contains two CE4 elements which are binding sites for hnRNP A1. B, incubation of the pre-mRNAs in HeLa extracts was for 2 hours in the presence of different percentages of DMSO (0, 0.8, 1.6, 2.4%). Labeled RNA products were fractionated on a denaturing 11% polyacrylamide gel;

FIG. 2 shows that DMSO does not affect the activity of hnRNP A1. A, HeLa extracts lacking or containing 2.4% DMSO were supplemented with recombinant hnRNP A1 proteins (0.125, 0.25, 0.5 and 1 μg) and splicing of the C5′ 4/4 pre-mRNA was monitored. B, diagram depicting the A1-mediated stimulation of distal 5′ splice site usage in extracts lacking or containing DMSO. The almost identical slopes suggest that the activity of recombinant A1 is not affected by DMSO.

FIG. 3 shows that DMSO affects 3′ splice site selection. A, structure of the pre-mRNAs used to assay modulation of 3′ splice site selection. C3′−/− is derived from the hnRNP A1 gene (Blanchette, M. and Chabot, B. 1999, supra). The NCAM 3′ pre-mRNA is a hybrid pre-mRNA containing the 5′ splice site of exon 7, the 3′ splice site of NCAM alternative exon 18 and the 3′ splice site of adenovirus L2 exon. B, in vitro splicing assays of model pre-mRNAs. Incubation was for 2 hours in HeLa extracts containing increasing percentages of DMSO (0, 0.8, 1.6, 2.4% in lanes 1-4, and 0, 0.8, 1.6, 2.4, 3.2% in lanes 5-9). Labeled RNA products were fractionated on denaturing 11% (for C3′ −/−) or 6.5% (for NCAM3′) polyacrylamide gels;

FIG. 4 shows that DMSO rescues splicing in a HeLa S100 extract. Splicing reactions were performed in a HeLa nuclear extract (NE) and in a HeLa S100 extract. The extracts were incubated in the absence or in the presence of 3.2% DMSO. The S100 extract was also supplemented with 0.5 μg of recombinant ASF/SF2 protein (lane 5). The pre-mRNA substrate used was C5′ 4/4;

FIG. 5 shows that DMSO activates SR proteins. A, using the C5′ 4/4 pre-mRNA, the activity of the recombinant SR proteins GST-SRp30c was tested in the absence and in the presence of increasing concentrations of DMSO. The GST-SRp30c protein (0.5 and 1 μg) was pre-incubated in nuclear extract 15 min at 30° C. before adding the pre-mRNA and DMSO. B, diagram depicting the SRp30c-mediated reduction in distal 5′ splice site usage in extracts lacking or containing different concentrations of DMSO;

FIG. 6 shows that DMF and formamide also affect alternative splicing in a cell free HeLa nuclear extract. In vitro splicing assays with the model pre-mRNA C5′ 4/4 were carried out in HeLa extracts in the presence of different percentages of DMF or formamide (0, 1.6, 2.4, 3.2 and 4%). For comparison, splicing of the same pre-mRNA in a HeLa nuclear extract containing 4% DMSO is shown (lanes 6 and 12); and

FIG. 7 shows that DMF but not formamide can activate splicing in a HeLa S100 extract. Splicing reactions with the C5′ 4/4 pre-mRNA were set up in HeLa S100 extracts in the presence of increasing amounts of DMSO, DMF or formamide (0, 2.4, 3.2 and 4%). For comparison, a splicing reaction performed in a HeLa nuclear extract is shown (NE, lane 13).

Other objects, advantages and features of the present invention will become more apparent upon reading of the following non-restrictive description of preferred embodiments with reference to the accompanying drawing which is exemplary and should not be interpreted as limiting the scope of the present invention.

### DETAILED DESCRIPTION OF THE INVENTION

It is shown herein that DMSO and related compounds can control alternative RNA splicing directly. This direct link is demonstrated by the fact that DMSO affects the alternative splicing of pre-mRNAs when assayed in extracts prepared from human HeLa cells (i.e., a model in vitro splicing system). Thus, the effects observed must affect factors involved in alternative splicing since these effects cannot be occurring through membrane-mediated effects, such as transcription, translation, etc. (i.e. indirectly).

DMSO Affects Splice Site Selection In Vitro

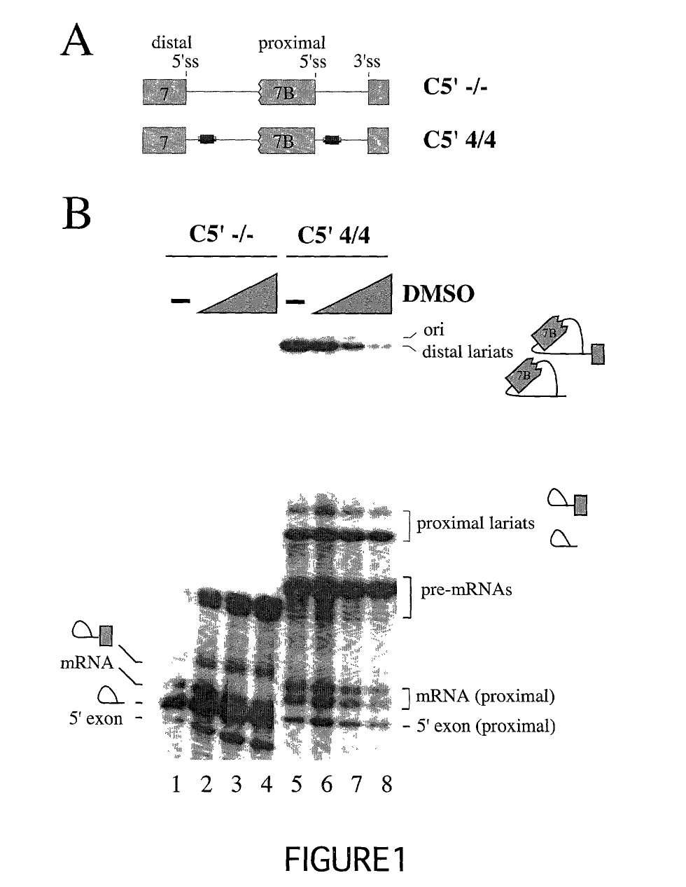

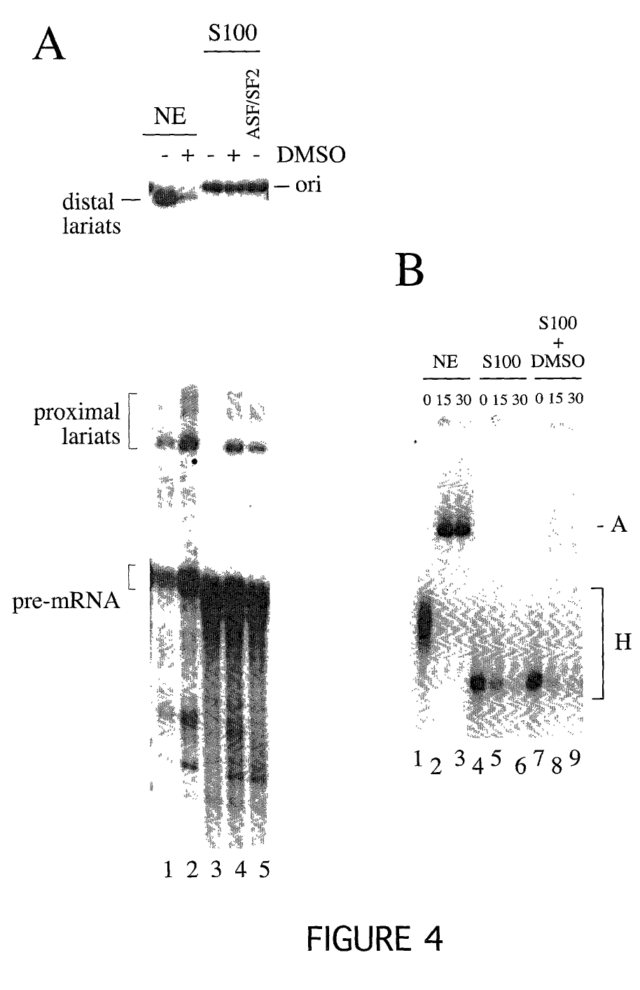

To assess whether DMSO can modulate splice site selection directly, the effect of adding DMSO to splicing reactions incubated in nuclear extracts prepared from HeLa cells was tested. A model pre-mRNAs derived from the hnRNP A1 alternative splicing unit (Blanchette, M. and Chabot, B., supra) was used. C5′ −/− contains two competing 5′ splice sites and a unique 3′ splice site (FIG. 1A). C5′ −/− is spliced almost exclusively to the proximal 5′ splice site (FIG. 1B, lane 1) as evidenced by the absence of distal lariat product near the top of the gel and the presence of proximal lariat products below the pre-mRNA. In contrast, the presence of A1 binding elements in C5′ 4/4 promotes efficient splicing to the distal 5′ splice site (lane 5), as shown by the presence of lariat products migrating near the top of the gel. The addition of DMSO at a final concentration of 0.8, 1.6 and 2.4% did not affect the splicing efficiency of C5′ −/− RNA, and 5′ splice site selection remained exclusively proximal (FIG. 1B, lanes 2-4). In contrast, DMSO promoted a strong reduction in the use of distal 5′ splice site in C5′ 4/4 pre-mRNA (lanes 6-8). The highest concentration of DMSO (lane 8) produced a 5-fold decrease in the use of the distal 5′ splice site. For example, compare the ratio of the intensity of the distal lariats band over the proximal lariat band in lane 5 and in lane 8. In some experiments, the reduction in distal 5′ splice site use was accompanied by an increase in the production of lariat products derived from the proximal 5′ splice site (e.g., see FIG. 4, lane 2).

The effect of DMSO on 5′ splice site selection was as strong on a pre-mRNA that was synthesized in the absence of cap analogue (not shown). Thus, the reduction in distal 5′ splice site usage was independent of the cap structure at the 5′ end of the pre-mRNA. DMSO also affected 5′ splice site selection in a model pre-mRNA carrying two copies of the 5′ splice site of exon 7. Identical effects were seen with DMSO solutions obtained from different suppliers, and the deionization of DMSO did not change its activity on 5′ splice site selection. Transient exposure of nuclear extracts to DMSO (i.e., incubation in the presence of DMSO followed by dialysis) did not affect 5′ splice site usage (not shown). Thus, DMSO needs to be present in the splicing mixtures to affect splice site selection.

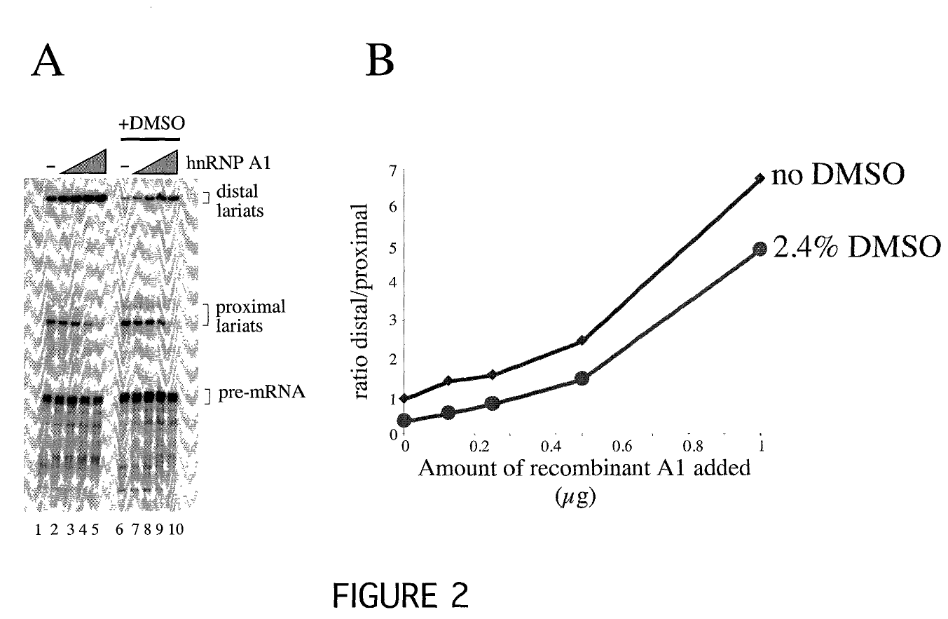

Because DMSO has a strong effect on the alternative splicing of a pre-mRNA carrying A1 binding elements (C5′ 4/4), it was then asked whether DMSO compromised the activity of the hnRNP A1 protein. We have shown previously that hnRNP A1 promotes distal 5′ splice site utilization on this pre-mRNA (Blanchette, M. and Chabot, B., 1999, supra). In nuclear extracts containing DMSO, the addition of hnRNP A1 efficiently shifted selection toward the distal 5′ splice site (FIG. 2A, lanes 6-10). The effect was specific since the addition of similar amounts of GST or gene 32 protein had no effect (not shown). Notably, the profile of stimulation obtained with increasing amounts of recombinant A1 was similar to the profile obtained in a nuclear extract lacking DMSO (FIG. 2A, lanes 1-5; compare the slopes in FIG. 2B). Because the activity of recombinant hnRNP A1 is not compromised by the presence of DMSO, DMSO is unlikely to affect the activity of the endogenous A1 proteins.

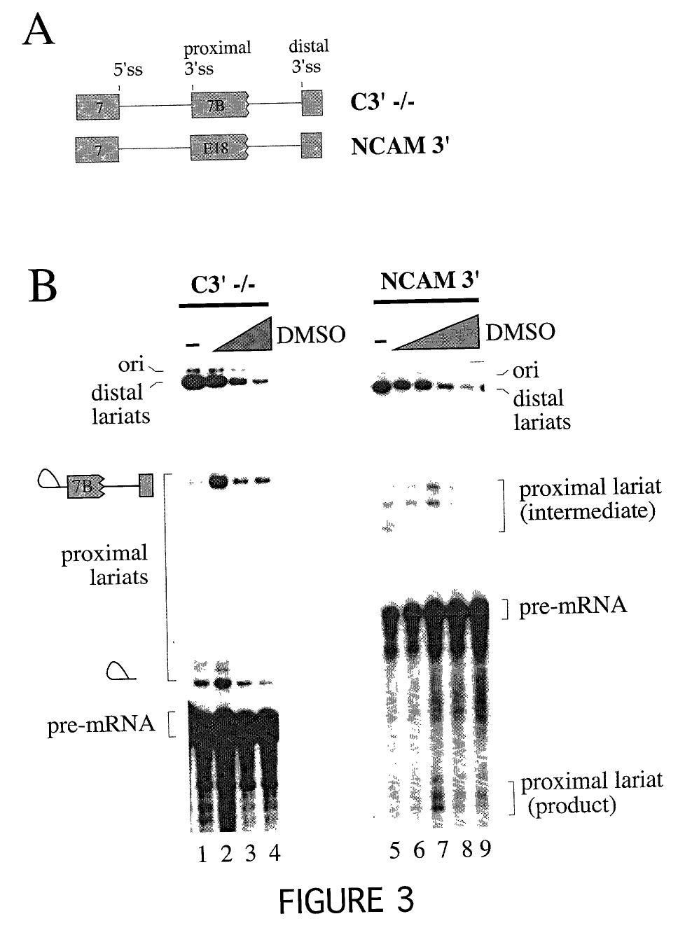

To address whether DMSO has a similar activity on 3′ splice site selection, a pre-mRNA (C3′ −/−; FIG. 3A) which is spliced predominantly to the distal 3′ splice site (FIG. 3B, lane 1) was tested. As shown for 5′ splice site selection, C3′ −/− splicing was sensitive to increasing amounts of DMSO (FIG. 3B, lanes 2-4). At the highest concentration of DMSO (lane 4), more than 50% of splicing occurred at the proximal 3′ splice site. A derivative of C3′ −/− in which the central portion was substituted for the 3′ splice site region and a portion of NCAM alternative exon 18 (NCAM3′ RNA) was also tested. Although splicing of NCAM3′ RNA was less sensitive to DMSO than C3′ −/−, DMSO promoted a stronger reduction in the use of the distal 3′ splice site as compared to the proximal 3′ splice site (FIG. 3B, lanes 5-9). Alternative splicing of a beta-globin pre-mRNA carrying duplicated 3′ splice sites was also affected by DMSO (data not shown).

DMSO Activates SR Proteins

The effect of DMSO on splice site selection is reminiscent of the activity of SR proteins which tend to activate splicing of the proximal pair of splice sites. Although DMSO did not stimulate overall splicing activity in nuclear extracts (FIG. 4, lanes 1 and 2), it was then tested whether DMSO could mimic the generic splicing activity of SR proteins. This activity was initially defined by the capacity of SR proteins to activate splicing in a HeLa S100 extract, either as a mixture of SR proteins or individually. U2AF⁶⁵ also activates splicing when added to a HeLa S100 extract. Surprisingly, the addition of DMSO to a HeLa S100 extract stimulated splicing as efficiently as the addition of the recombinant SR protein ASF/SF2 (FIG. 4, lanes 3-5) as seen by the comparable level of proximal lariats observed in lanes 4 and 5. The addition of DMSO to a S100 extract also stimulated the formation of complexes, as judged by native gel analysis (data not shown). These results suggest that DMSO increases the activity of residual amounts of SR or U2AF proteins in the S100 extract. Thus, DMSO and related compounds not only affect alternative splicing but can also affect generic splicing. The level of DMSO-dependent splicing stimulation varied considerably in different preparations of S100 extract. Although DMSO and recombinant ASF/SF2 restored splicing activity in a similar manner, splicing to the distal 5′ splice site was not detected, as is the case in a nuclear extract (lanes 1 and 2, see distal lariats). Our group has shown previously that distal 5′ splice site selection on this pre-mRNA requires hnRNP A1 (Blanchette, M. and Chabot, B., 1999, supra). The failure to activate distal 5′ splice site use is probably due to the fact that S100 extracts contain small amounts of hnRNP A/B proteins as compared to nuclear extracts (not shown).

In any event, these results show that the inherent variation of splicing can be normalized by the addition of DMSO (or related compound). For example, the splicing activity between different S100 extracts can be normalized by the addition of DMSO or related compound. In addition, the alternative splicing profile of an extract (which often varies between nuclear extracts) can also be normalized with DMSO or related compounds. For example, if two nuclear extracts use distal splice sites with different frequencies, the ratio of distal/proximal splice site selection can be normalized using DMSO (or the like) in order to shift splicing towards the proximal site.

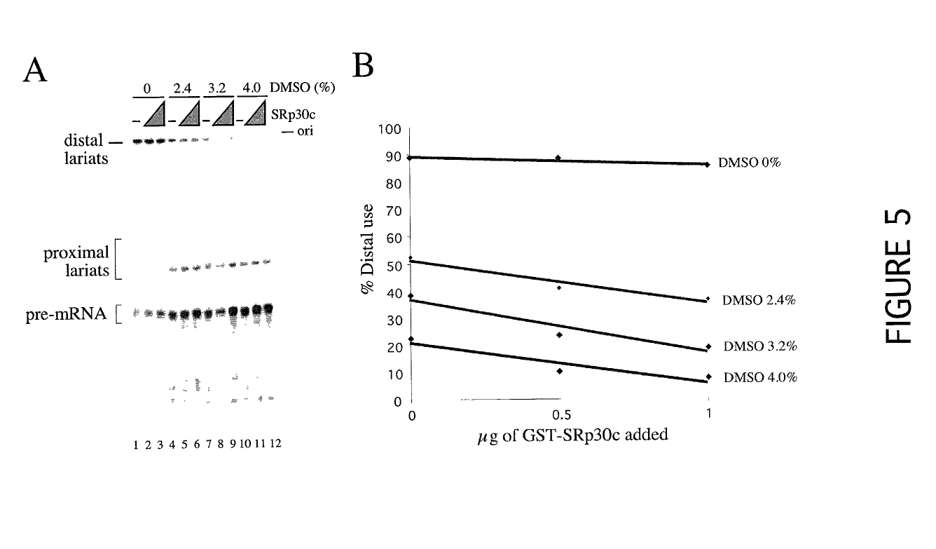

The above results suggest that DMSO may exert their splicing effect through an activity of SR proteins. To further examine this possibility, the effect of adding DMSO to splicing reactions pre-incubated with a recombinant SR protein was tested. At the concentrations used and in the absence of DMSO, the recombinant SR protein GST-SRp30c had little effect on 5′ splice site selection when using the C5′ 4/4 pre-mRNA (FIG. 5, lanes 1-3). However, in the presence of DMSO, the same amount of GST-SRp30c stimulated proximal 5′ splice site utilization (lanes 4-12) as observed by a shift in the ratio of distal lariats/proximal lariats. Thus, the simultaneous addition of DMSO and SR produced a shift toward proximal use that was greater than the sum of their individual contribution. Because recombinant SR proteins display more activity in the presence of DMSO, a similar effect on the endogenous SR proteins may appear to be responsible for the activity of DMSO in nuclear extracts.

DMF and Formamide also Modulate Splice Site Selection

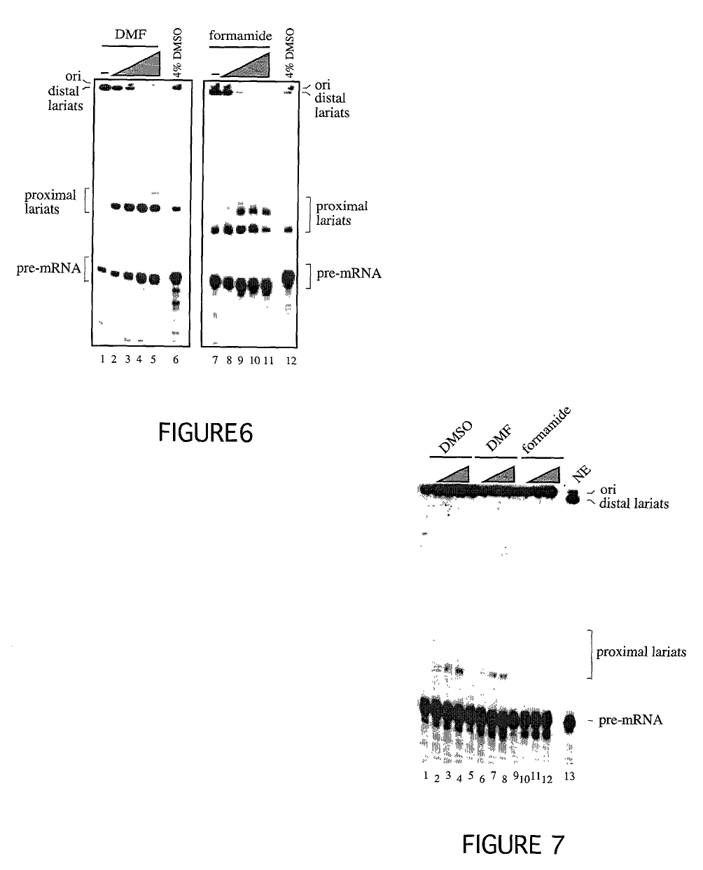

To understand the chemical basis for the activity of DMSO in alternative splicing, other solvents were tested. Interestingly, at equivalent percentages, both DMF and formamide were at least as active as DMSO at modulating 5′ splice site selection (FIG. 6). Surprisingly, although DMF and formamide shared with DMSO the ability to modulate 5′ splice site selection, formamide was unable to activate splicing in a HeLa S100 extract (FIG. 7).

The addition of DMSO to nuclear extracts can have strong effects on splice site selection while having minimal effects on the efficiency of splicing. In contrast, the addition of DMSO to a splicing-deficient HeLa S100 extract stimulated splicing in a manner reminiscent of the activity of SR proteins. The effect of DMSO on splice site selection was also similar to the activity of SR proteins since DMSO shifted selection towards the proximal pair of splice sites. Consistent with the notion DMSO stimulates the activity of SR proteins, it was found that the combination of DMSO and SRp30c produces a shift that is greater than the sum of their individual contribution. Thus, a general stimulation in the activity of all endogenous SR proteins most probably explains why DMSO influences splice site choice in vitro. Likewise, the addition of DMSO to a S100 extract may stimulate the residual amounts of SR proteins present in this extract.

Although the results presented herein strongly suggest that DMSO affects the activity of SR proteins, the mechanism by which SR proteins become activated remains unclear. Western analysis using an antibody that recognizes phosphorylated epitopes on SR proteins revealed no change in the overall and relative abundance of phosphorylated SR proteins upon incubation with DMSO (data not shown). Moreover, DMSO did not affect the binding of SR proteins to a purine-rich RNA splicing enhancer element (data not shown). DMSO also did not modify the solubility of SR proteins when extracts were incubated with increasing concentrations of MgCl₂ (data not shown). Although DMSO is regarded as a relatively inert solvent for pharmacological applications, it improves the solvation of cations and stimulates nucleophilic reactions. Of note, DMF and formamide share this chemical property with DMSO. Thus, DMSO may improve the solvation of positive charges on proteins. This may influence the structure at the surface of proteins and facilitate ionic contacts between charged domains of interacting proteins. Consistent with this view, modulation of 5′ splice site selection in vitro is known to be sensitive to the ionic conditions of the reaction (Schmitt, P et al., 1987, Cell 50:31). Splicing proteins that carry charged domains include SR and U2AF proteins which have RS-domains rich in positively and negatively charged amino acids (arginines and phosphorylated serines, respectively). Interactions between the RS-domain containing proteins ASF/SF2, U1 snRNP-70 kD, and U2AF³⁵ have been proposed to occur early during spliceosome assembly (Wu, J. Y., and Maniatis, T. 1993, Cell 75:1061). Moreover, these interactions are sensitive to the phosphorylation state of ASF/SF2 (Xiao, S. H., and Manley, J. L. 1998, EMBO J. 17:6359). Without being limited to a particular theory, DMSO may thus activate splicing in a S100 extract by improving the quality of the ionic interactions between residual amounts of SR and U2AF proteins. Since the amount and activity of these proteins are in excess in a nuclear extract, this would explain why DMSO stimulates generic splicing in a S100 but not in a nuclear extract.

Even though nuclear extracts contain sufficient amounts of SR proteins for generic splicing, their activity in splice site selection is not maximal since adding more SR proteins can have a strong effect on the selection of splice sites (Ge, H. and Manley, J. L. 1990, Cell 62:25; Krainer, A. R., et al. 1990. Cell 62:35). Since a similar effect can be obtained by adding kinases that phosphorylate the RS domains of SR proteins (Prasad, J et al., 1999, Mol Cell Biol 19:6991), the profile of charged residues at the surface of SR proteins is crucial for their activity as splicing regulators. Moreover, the requirement for charged residues at the surface of SR proteins appears different for generic and alternative splicing because dephosphorylation of ASF/SF2 is essential for constitutive splicing, but is not required for the protein to function as a splicing regulator (Xiao, S. H., and Manley, J. L. 1998, supra). Thus, DMSO may affect the presentation of charged residues that are important for the activity of SR proteins in splice site selection. Since DMSO and DMF activate splicing in a HeLa S100 extract, DMSO and DMF may also affect the presentation of different residues that are important for generic splicing. In contrast, because formamide affect splice site selection but cannot activate a S100 extract, formamide may only affect the presentation of residues that play a role in splice site selection.

The results presented herein raise the possibility that the documented effect of DMSO on cell differentiation may be caused, at least in part, by changes in the activity of SR proteins, which in turn affect splice site selection. This conclusion is supported by the observation that DMF can mimic the effect of DMSO both in differentiation assays (Blau, H. M., and Epstein, C. J. 1979, Cell 17:95; Pise, C. A., et al. 1992, J Gen Virol 73:3257; Shen, Q. et al. 1994, Blood 84:3902; Hoosein, N. M., et al. 1988, Exp Cell Res 175:125), and in splicing assays in vitro. Depending on the cell types, DMSO can either promote or block differentiation or apoptosis. These opposite outcomes may be explained if different subsets of pre-mRNAs are expressed in different cell types. For example, alternative splicing is often used to control the production of proteins involved in programmed cell death such as Fas, Bcl-2, Bax, and Ced-4. Hence, DMSO may alter the alternative splicing of a pre-mRNA to favor the production of a repressor protein in one cell type, whereas an inducer may be produced from another gene in a different cell type.

Whatever the precise mechanism of action of DMSO, DMF, formamide and related compounds on splicing and/or splice site selection, the present invention opens the way to numerous methods of modulating, selecting, and identifying splice site units and to dissect the structure function relationship of SR proteins in splicing and splice site selection. The identification of the stimulating effect of DMSO, DMF, formamide and the like on splicing also enables the designing of splicing kits and of methods of normalizing splicing extracts.

The present invention is illustrated in futher detail by the following non-limiting examples.

### EXAMPLE 1

### Preparation of the S100 and Nuclear Extracts

The preparation of nuclear extracts and S100 extracts is well known in the art. The extracts of the present invention were prepared using the protocol of Dignam et al. 1983. (Nucl. Acids. Res. 11, 1475-1489).

### EXAMPLE 2

### Preparation of the Pre-mRNA Transcripts and Splicing Assays

These procedures are also standard in the field and can be found, for example, in Chabot B., 1994 (RNA processing-A Practical Approach. Volume 1, Chapter 1. Oxford University Press. pp. 1-29; Eperon et al., 1994 (RNA processing-A Practical Approach. Volume 1, Chapter 1. Oxford University Press. pp. 57-101).

A specific example of a particular splicing assay is as follows:

Splicing extract=7 μl (already containing DMSO)

Splicing buffer

0.5 μl of rATP 12.5 mM

0.5 μl of MgCl₂ 80 mM

0.5 μl of creatine-phosphate 0.5M

2.5 μl of PVA 13%

0.25 μl of DTT 100 mM

0.25 μl of RNAguard or RNasin

0.5 μl of H₂O

Add 0.5 μl of labeled pre-mRNA in H₂O.

Incubation of the mixture at 30° C., and analysis of the transcripts was carried-out as in Chabot 1994 (supra).

### EXAMPLE 3

### Binding Assays

Nuclear extracts and S100 extracts were prepared according to the procedure of Dignam et al., 1983, supra (see Example 1). The splicing reactions were set-up according to Krainer et al. 1985 (Cell 42, 725-736) and Eperon et al., 1994, supra. Labeled pre-mRNAs were prepared as described in Chabot 1994, supra. DMSO was added to splicing reactions before incubation to obtain a final concentration of DMSO between 1% to 5%. (The optimal concentration of DMSO depends on the extract and has to be determined empirically for each extract). The mixtures were incubated at 30° C. between 5 min to 1 hour and the splicing complexes were fractionated on gels using the following procedure:

a) a 4 μl aliquot was removed from the reaction mixture and 1 μl of heparin (1 mg/ml) was added.

b) the mixture was put on ice, and 0.5 μl of loading buffer (50% glycerol, 1% bromophenol blue, 1% xylene cyanol) was added.

c) the samples were then loaded onto a 4% polyacrylamide gel (acrylamide/bis-acrylamide 80:1) in 50 mM Tris-glycine which had been pre-electrophoresed at 190 volts for 30 min.

d) gel electrophoresis was performed at 190 volts for 3-4 hours at room temperature.

e) following separation, the gel was autoradiographed.

Of course, the splicing/alternative splicing profile of the pre-mRNA could be determined in parallel in order to further dissect the mechanistic details of complex formations and modulation of splicing.

### EXAMPLE 4

### DMSO Affects Alternative Splicing In Vivo in Mouse N2a Cells

Mouse neuroblastoma N2a cells were cultured at 37° C. in DMEM supplemented with 10% bovine calf serum. Confirming previously studies (Pollerberg et al. 1986, Nature 324(6096), 462; 31; Tacke et al. 1991, Genes Dev. 5, 1416), the treatment of N2a cells with 2% DMSO for 48 hrs was shown to improve the frequency of inclusion of the neuro-specific exon 18 in the NCAM pre-mRNA. For treatment with DMSO, medium containing 2% bovine calf serum was used. Following treatment, total RNA was isolated and a Rnase T1 protection assay was performed using a uniformly labeled 530 nt NCAM antisense RNA probe. Exon 17/exon19 splicing yielded a 303 protected fragment while the inclusion of exon 18 produced a 452 nt fragment. Products were resolved on a 5% denaturing acrylamide gel. A strong increase in the abundance of the mRNA carrying exon 18 was observed (not shown).

### EXAMPLE 5

### DMSO Affects Alternative Splicing In Vivo in Human HeLa Cells

Human HeLa cells were cultured at 37° C. in DMEM supplemented with 10% bovine calf serum. A similar effect, as observed in Example 4, was observed on the hnRNP A1 pre-mRNA. In this case, the inclusion frequency of alternative exon 7B following the treatment of HeLa cells for 5 hours with 5% DMSO was observed. A RT-PCR assay was used to amplify products corresponding to exon 7B inclusion. Although the effect was less dramatic than for the NCAM pre-mRNA, DMSO treatment significantly improved the inclusion of exon 7B (not shown).

Although the present invention has been described hereinabove by way of preferred embodiments thereof, it can be modified, without departing from the spirit and nature of the subject invention as defined in the appended claims.

What is claimed is:

1. A method to modulate splicing and/or alternative splicing in vitro comprising an administration to a cell or extract thereof of an effective amount of a polar aprotic solvent, whereby said effective amount modulates splicing and/or alternative splicing as compared to an untreated cell or extract.

2. The method of claim 1, wherein said solvent is selected from DMSO, DMF and formamide.

3. The method of claim 2, wherein said solvent is DMSO or DMF and said modulation is effected on a nuclear extract.

4. The method of claim 4, wherein said effective amount modulates alternative splicing.

5. The method of claim 1, wherein said modulation is effected through an effect on at least one SR protein.

6. A method of modulating the splicing and/or alternative splicing activity of a SR protein comprising an administration to a cell or extract thereof containing said SR protein of an effective amount of a polar aprotic solvent, whereby said effective amount modulates said activity of said SR protein as compared to a non-treated cell or extract.

7. The method of claim 6, wherein said solvent is selected from DMSO, DMF and formamide.

8. The method of claim 7, wherein said solvent is DMSO or DMF and said modulation is effected on a nuclear extract.

9. The method of claim 8, wherein said effective amount modulates said alternative splicing activity of said SR protein.

10. A splicing kit comprising:
a) a container containing a splicing and/or alternative splicing-competent extract;
b) a second container containing a splicing and/or alternative splicing buffer; and
c) a polar aprotic solvent.

11. The kit of claim 10, wherein said polar aprotic solvent is selected from DMSO, DMF, formamide, HMPA, N-methylformamide, nitromethane, acetone, and acetonitrile.

12. The kit of claim 11, wherein said solvent is DMSO.

13. The kit of claim 11, wherein said wherein said solvent is present in said first container.

14. The kit of claim 12, wherein said extract is a nuclear extract whose splicing activity is normalized by said DMSO.

15. The kit of claim 11, wherein said solvent is contained in a third container.

16. A method to normalize a splicing and/or alternative splicing activity of an extract comprising an addition thereto of an effective amount of a polar aprotic solvent, whereby said effective amount normalizes splicing and/or alternative splicing as compared to an untreated extract.

17. The method of claim 16, wherein said solvent is selected from DMSO, DMF, formamide, HMPA, N-methylformamide, nitromethane, acetone, and acetonitrile.

18. The method of claim 17, wherein said solvent is DMSO or DMF and said normalization is effected on a nuclear extract.

19. The method of claim 18, wherein said effective amount modulates alternative splicing.

20. The method of claim 16, wherein said normalization is effected through an effect on at least one SR protein contained in said extract.
